(19) United States
(12) Patent Application Publication
(10) Pub. No.: US 2004/0055034 A1
(43) Pub. Date: Mar. 18, 2004

(54) Multiple disease resistant early flowering cucumber variety

(76) Inventors: Margaret M. Jahn, Lansing, NY (US); Henry M. Munger, Ithaca, NY (US)

Correspondence Address: Michael L. Goldman, Esq., NIXON PEABODY LLP, Clinton Square, P.O. Box 31051, Rochester, NY, 14603-1051, US

(21) Appl. No.: 10/246,269

(22) Filed: Sep. 18, 2002

(51) Int. Cl. A01H 1/00; C12N 15/82; A01H 5/00; C12N 5/04

(52) U.S. Cl. 800/279; 800/307; 435/419

(57) ABSTRACT

The present invention relates to a multiple disease resistant, early-flowering inbred cucumber seed, and methods for producing such a multiple disease resistant, early-flowering, inbred cucumber seed. The present invention is further directed to plant seeds, plants, pollen, and ovules produced by carrying out the method of the present invention.

This invention was developed with government funding under Hatch Grant, No. 149485. The U.S. Government may have certain rights.

### FIELD OF THE INVENTION

The present invention relates to C. sativus L. inbred seeds and plants having early flowering and multiple disease resistance, methods of producing early flowering C. sativus L. seeds and plants having multiple disease resistance, and early flowering, multiple disease resistant seeds and plants produced by using the inbred C. sativus L. as a parent in a breeding program.

### BACKGROUND OF THE INVENTION

Cucumbers originated in India where they have been cultivated for 3000 years. The cucumber (Cucumis sativus) is a member of the Cucurbitaceae family, also known as the gourd family. This family consists of more than 100 genera, but only three are of commercial importance in the U.S.: Cucumis (cucumber and muskmelon), Citrullis (watermelon), and Cucurbita (pumpkin and squash).

Cucurbits are generally annuals that are extremely intolerant of cold weather. They are grown mainly for their fruits, which are derived from a single ovary containing many ovules or seeds. In some parts of the world, flowers and leaves of some species are also used for food. Among vegetable crops, the cucurbits are somewhat different in that they bear different kinds of flowers on the same plant. In most cucurbits, the pollen is rather sticky and heavy, and pollination by insects is required. Cucumbers are both a leading commercial crop and a popular home garden vegetable. Cucumber cultivars are usually classified according to their intended use as fresh market slicers, pickles, or greenhouse cucumbers.

The major portion of the commercial cucumber crop in the U.S. is processed as pickles, with Michigan, North Carolina, Texas, California, and Wisconsin the leading states for pickle production. Processing cucumber yields average about 5 tons per acre in the U.S., and range from about 4 tons per acre in the east to over 18 tons per acre in California. Approximately 40% of the pickling crop is fresh-packed, 15% is refrigerated, and 40-45% is brined. Cucumbers for pickling are usually grown under contract, so profits are directly related to yield per acre.

Florida, the Carolinas, and Texas lead in fresh market production. Average yields for fresh market slicers picked by hand range from 3 tons per acre in parts of the eastern U.S. to over 16 tons per acre in California; top yields can be much higher. Fresh market cucumbers produced early in the marketing season are a high-value crop in most areas of the U.S.

Cucurbits are affected by a large number of diseases caused by bacteria, fungi, and viruses. Among the most common cucumber diseases are angular leafspot disease, caused by the bacterium Pseudomonas lachrymans; cucumber mosaic virus (“CMV”), which is found in many areas of the country, overwinters on seeds and roots, and is spread by insects; and scab, caused by the fungus Cladosporium cucumerinum, which produces sunken, dark brown spots on the fruits. In addition, cucumbers may be infected by target leafspot disease (“TLS”) caused by Corynespora cassiicola, zucchini yellow mosaic virus (“ZYMV”), watermelon mosaic virus (“WMV”), papaya ringspot virus (“PRSV”), downy mildew, anthracnose, root knot (caused by nematodes), damping-off, powdery mildew, Alternaria and Cercospora leaf blights, gummy stem blight, and black rot. Viral diseases are often the most destructive and the most difficult to control.

Disease control is critical in the large-scale production of high quality slicing cucumbers. A preventative program generally combines the use of cultural practices, genetic resistance, and chemical control to limit the establishment, spreading, and survival of cucumber pathogens. However, cultural practices require frequent crop rotation, optimal soil conditions, and exacting planting conditions to avoid the spread of the many fungal, bacterial, and nematode pathogens that survive in old crop debris and in soil. These soil-borne pathogens can be carried into clean fields by the movement of workers or equipment. Improper irrigation and tillage can also create conditions which encourage the survival and spread of cucurbit pathogens which reside in soil.

Also difficult to control are diseases carried on seed, such as anthracnose, angular leafspot disease, and gummy blight. Control of these diseases requires pre-planting fungicide seed treatment. Chemical fungicide treatment is expensive, time-consuming, and potentially hazardous to the health of those who apply it. As for genetic control, only a limited number of disease-resistant varieties of cucumber have been bred to date, and those often are resistant to just a few of the many pathogens that can infect and devastate a cucumber crop.

The present invention is directed to overcoming these and other deficiencies in the art.

### SUMMARY OF THE INVENTION

The present invention relates to an early flowering inbred cucumber seed designated ‘Marketmore 2000’ having resistance to target leafspot disease, zucchini yellow mosaic virus, watermelon mosaic virus, and papaya ringspot virus, a sample of said seed having been deposited under ATCC accession number ______.

The present invention also relates to a method for producing a multiple disease resistant, early-flowering inbred cucumber seed, a sample of said seed having been deposited under ATCC accession number ______. This method involves providing a first inbred C. sativus L. cucumber plant line which is resistant to target leafspot disease, zucchini yellow mosaic virus, watermelon mosaic virus, and papaya ringspot virus, and a second inbred C. sativus L. cucumber plant line which is early flowering and resistant to zucchini yellow mosaic virus, watermelon mosaic virus, and papaya ringspot virus. The first and second inbred C. sativus L. cucumber plant lines are crossed to yield an F1 hybrid C. sativus L. cucumber plant which is early flowering and resistant to target leafspot disease. This F1 hybrid cucumber plant is then backcrossed to yield an early flowering inbred cucumber plant line which is resistant to target leafspot disease, zucchini yellow mosaic virus, watermelon mosaic virus, and papaya ringspot virus; and the seeds are harvested from-the plant.

The present invention also relates to pollen, ovules, seeds, and tissue culture derived from the multiple disease resistant, early-flowering C. sativus L. cucumber seeds and plants produced according to the methods of the present invention.

The use of disease-resistant plant varieties is an economical means of controlling disease in vegetable crops. Therefore, the development of a variety of slicing cucumber with an improved scope of disease resistance is highly desirable. Also desirable in cucumbers is the trait of early pistillate flowering (“earliness”). Because early flowering is related to early maturation, a cucumber line with the characteristic of earliness has fruit ready for market earlier in the growing season than other cultivars, and thus has a commercial advantage.

The present invention is useful in developing multiple disease resistant, commercially appealing C. sativus L. plants and plant parts.

### BRIEF DESCRIPTION OF THE DRAWINGS

FIG. 1 is a diagram of the origin and breeding history of the ‘Marketmore 2000’ cucumber cultivar. Key: M=Marketmore; BC=Backcross; SP=Self-Pollinate; TLSR=Target Leafspot Disease Resistance. The exponent in ‘M97’ indicates number of total crosses.

FIGS. 2A-B are photographs demonstrating the prolific flowering of the ‘Marketmore 2000’ line, shown in FIG. 2A, in comparison to its parental line, ‘Marketmore 97,’ shown in FIG. 2B.

### DETAILED DESCRIPTION OF THE INVENTION

The following definitions apply to the present application:

The terms “cultivar” and “variety” are used interchangeably in the present application.

The term “crossing” refers to the mating of two parent plants.

The term “backcrossing” refers to a process in which a breeder repeatedly crosses hybrid progeny, for example a first generation hybrid (F1), back to one of the parents of the hybrid progeny.

“Earliness” and “early flowering” are used interchangeably, and as used herein refer to early pistillate flowering, i.e., early production of flowers, measured in days to first appearance of pistillate flowers.

The term “monoecious” refers to plants in which staminate (male) and pistillate (female) reproductive organs are borne on separate flowers but on the same individual.

The term “F1 hybrid” refers to the first generation progeny of the cross of two non-isogenic plants.

The term “genotype” refers to the genetic constitution of a cell or organism.

“Resistance” as used herein means resistance to a disease, and not necessarily to the pathogen that caused the disease.

The present invention relates to a method for producing an early flowering, multiple disease resistant inbred cucumber seed, known as ‘Marketmore 2000’ (“M2000”), of which a seed sample has been deposited under ATCC Accession No. ______. This involves providing a first inbred C. Sativus L. cucumber plant line which is resistant to target leafspot disease, zucchini yellow mosaic virus, watermelon mosaic virus, and papaya ringspot virus, and a second inbred C. Sativus L. cucumber plant line having the characteristics of early flowering and resistance to zucchini yellow mosaic virus, watermelon mosaic virus, and papaya ringspot virus. The first and second inbred lines are crossed to yield an F1 early flowering cucumber seed with resistance to target leafspot disease. This F1 hybrid cucumber plant is then backcrossed to yield an early flowering inbred cucumber plant line which is resistant to target leafspot disease, zucchini yellow mosaic virus, watermelon mosaic virus, and papaya ringspot virus, and the seeds are harvested from the plant.

In one aspect of the present invention, the first parent for this cross is a cucumber cultivar resistant to target leafspot disease, zucchini yellow mosaic virus, watermelon mosaic virus, and papaya ringspot virus, while the second parent is resistant to zucchini yellow mosaic virus, watermelon mosaic virus, and papaya ringspot virus, and exhibits early pistillate flowering.

In another aspect of the present invention, the first parent cultivar may be resistant to cucumber scab, powdery mildew, downy mildew, cucumber mosaic virus, Alternaria leaf blight, and Ulocladium leafspot disease in addition to target leafspot disease and potyvirus.

In a further aspect of the present invention, the second parent cultivar may be resistant to cucumber scab, powdery mildew, downy mildew, and cucumber mosaic virus in addition to potyvirus, and exhibit early and prolific flowering.

An example of a suitable first parental cultivar of the present invention is ‘Marketmore 97’ (‘M97’), a slicing variety of C. sativus L. ‘M97’ is characterized as monoecious, with uniform color, having non-bitter fruit, and resistant to the potyviruses (ZYMV, WMV, PRSV), CMV, cucumber scab, powdery and downy mildew, Alternaria leaf blight, Ulocladium leafspot disease, and TLS.

An example of a suitable second parental cultivar of the present invention is ‘Marketmore 421,’ (‘M421’) a slicing variety of C. sativus. ‘M421’ is characterized as monoecious, with uniform color and bitter fruit. ‘M421’ is resistant to the potyviruses (ZYMV, WMV, PRSV), CMV, cucumber scab, downy mildew and powdery mildew.

The present invention also encompasses the use of germplasm from the inbred multiple disease resistant, early flowering plant of the present invention in any breeding program to yield the multiple disease resistant early-flowering cucumber seeds or plant of the present invention, and any inbred multiple disease resistant, early flowering seeds, plants, and plant parts derived therefrom.

The present invention further relates to the inbred TLS, CMV, ZYMV, WMV, PRSV, cucumber scab, powdery mildew, downy mildew, Alternaria leaf blight, and Ulocladium leafspot disease resistant, early-flowering cucumber seed produced by the method of the present invention. After a multiple disease resistant, early-flowering plant of the present invention has been tested and individuals identified and selected for optimum performance, the inbred seed of the present invention is produced and harvested from the inbred multiple disease resistant, early-flowering plant of the present invention.

From the inbred resistant seed, it is possible to grow an early flowering, multiple disease resistant cucumber plant having all the physiological and morphological characteristics of the early flowering, multiple disease resistant hybrid cucumber plant described herein. Multiple disease resistant, early-flowering hybrid cucumber plants of the present invention can be readily produced from the multiple disease resistant, early-flowering inbred seed of the present invention using growing techniques and conditions known to those skilled in the art.

As used herein, the term “plant” includes plant cells, plant protoplasts, plant cell tissue culture from which plants can be regenerated, plant calli, plant clumps, and plant cells that are intact in plants or parts of plant, such as flowers, embryos, pollen or ovules. Therefore, the present invention also relates to parts of an early flowering, multiple disease resistant hybrid cucumber plant of the present invention, especially the pollen and ovule of such a plant, from which a TLS, CMV, ZYMV, WMV, PRSV, cucumber scab, powdery mildew, downy mildew, Alternaria leaf blight, and Ulocladium leafspot disease resistant, early-flowering cucumber plant can be produced. Therefore, early flowering, multiple disease resistant C. sativus inbred and hybrid plants, seeds, and plant parts made using a method of the present invention under any traditional breeding methods, including but not limited to, pedigree breeding, selling, intercrossing, and backcrossing, are also encompassed by the present invention.

Plant breeding programs are designed to produce new cultivars with superior qualities by the deliberate crossing of parent plants with the most highly desirable traits. Resistance to disease is a highly desirable trait, therefore, disease resistance is often one goal of a plant breeding program. How that goal is accomplished may vary, depending on the plant being bred. For example, in self-pollinated crops, the development of disease-resistant varieties can routinely be accomplished with the backcross and pedigree methods.

Backcrossing, for example, can be used in conjunction with the present invention to improve an inbred line and a hybrid which is made using those inbreds. Backcrossing is especially useful for transferring a specific desirable trait from one inbred or source to an inbred that lacks that trait. Backcross methods rapidly introgress specific resistance genes from a donor parent into a desirable variety, concurrently reconstructing the original variety. The selection of a suitable recurrent parent is an important step for a successful backcrossing procedure. The goal of a backcross is to alter or substitute a single trait or characteristic in the original inbred plant. This is accomplished by modifying or substituting a single gene of the recurrent inbred with the desired gene from the recurrent parent, while retaining essentially all of the rest of the desired physiological and morphological characteristics of the original inbred. For example, this can be accomplished by first crossing a superior inbred (recurrent parent) to a donor inbred (non-recurrent parent), that carries the appropriate gene(s) for the trait in question. The recurrent parent typically includes many commercially important traits, while the non-recurrent parent includes a resistance trait, as is the case for the TLS resistant cucumbers. The progeny of this cross are then mated back to the superior recurrent parent followed by selection in the resultant progeny for the desired trait to be transferred from the non-recurrent parent. Through backcrossing, a new variety is produced quickly, requires minimal testing, and is nearly isogenic with the original. Backcross breeding has been used extensively for breeding disease-resistant cultivars and, therefore, is highly suitable for use in the present invention. (Nickell et al., “Registration of L84-5873 and L84-5932 Soybean Germplasm Lines Resistant to Brown Stem Rot,” Crop Sci. 32:835 (1992), which is hereby incorporated by reference in its entirety).

Also suitable in the present invention for incorporating disease resistance are breeding methods such as pedigree, F2 selection, single seed descent, and the nested hierarchy method. Pedigree methods are useful when both parents are adapted and carry genes for resistance to different diseases. Compared with the backcross, a more genetically diverse germplasm base is developed, but the time to release a variety is much longer. The backcross and pedigree methods are forms of inbreeding that ultimately result in the development of pure lines. The level of homozygosity increases with each generation of self or backcrossing.

Disease resistant cucumbers, including, without limitation, the multiple disease resistant and early-flowering cucumbers of the present invention, can be used in a pedigree breeding program. Pedigree breeding starts with the crossing of two genotypes, each of which may have one or more desirable characteristics that is lacking in the other or which complements the other. If the two original parents do not provide all the desired characteristics, other sources can be included in the breeding population. In the pedigree method, superior plants are selfed and selected in successive generations. In the succeeding generations the heterozygous condition gives way to homogeneous lines as a result of self-pollination and selection. Typically, five or more generations of selfing and selection is practiced: F1→F2; F2→F3; F3→F4; F4→F5, etc. Variations of this generalized pedigree method are used, but all these variations produce a segregating generation which contains a range of variation for the traits of interest.

Thus, in general terms, pedigree breeding involves crossing two inbred lines to produce the non-segregating F1 generation, and self pollination of the F1 generation to produce the F2 generation that segregates for all factors for which the inbred parents differ. A hypothetical example of this process is set forth below.

Hypothetical Example of Pedigree Breeding Program:

Cross between two inbred lines that differ for alleles at six loci. The parental genotypes are:

| | |
|---|---|
| Parent 1 | AbCdeF/AbCdeF |
| Parent 2 | aBcDEf/aBcDEf, |

and the F1 from a cross between these two parents is:

| | |
|---|---|
| F1 | AbCdeF/aBcDEf |

Selfing the F1 will produce an F2 generation including the following genotypes:

| | |
|---|---|
| F2 | ABcDEf/abCdeF |
| F2 | ABcDef/abCdEF |
| F2 | ABcDef/abCdeF. |

Each inbred parent which is used in breeding crosses represents a unique combination of genes, and the combined effects of the genes define the performance of the inbred and its performance in hybrid combination. There is published evidence (Smith et al., “Similarities Among a Group of Elite Maize Inbreds as Measured by Pedigree, F₁ Grain Yield, Heterosis, and RFLPs,” Theor. Appl. Genet. 80:833-840 (1990), which is hereby incorporated by reference in its entirety) that each of the lines are different and can be uniquely identified on the basis of genetically-controlled molecular markers.

Mass and recurrent selections can be used to improve populations of either self-or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Elite inbred lines, that is, pure breeding, homozygous inbred lines, can be used as starting materials for breeding or as source populations from which to develop other inbred lines. These inbred lines derived from elite inbred lines can be developed using the pedigree breeding and recurrent selection breeding methods described earlier. As an example, when backcross breeding is used to create these derived lines in a melon plant breeding program, elite inbreds can be used as a parental line or starting material or source population and can serve as either the donor or recurrent parent.

A breeding program in accordance with the present invention will include a periodic objective evaluation of the efficiency of the chosen breeding procedure. Evaluation criteria include disease-resistance and other characteristics important in commercial cucumber crops, such as vigor, uniformity, earliness, and yield. Resistance as used herein means resistance to a disease, and not necessarily to the pathogen that caused the disease. Breeders select breeding lines for resistance to disease on the basis of reduced symptoms and better growth, seldom knowing whether the ‘resistance’ is brought about by some mechanism such as reduced replication or movement of the virus or by tolerance to the presence of the virus. It is convenient to speak of resistance to cucumber mosaic virus, for example, but more accurate to talk about resistance to the disease. Following initial evaluations, promising advanced breeding lines are thoroughly tested and evaluated against appropriate standards in the commercial target area. The best lines are potential new commercial cultivars.

For a breeder interested in improving the performance of a genotype under field conditions, yield and quality of the marketable portion of the plant are of primary concern. Therefore, specific phenotypic traits that are considered “desirable” characteristics are selected for in commercial breeding programs. The earliness trait of the cucumber plant of the present invention, for example, is a commercially important phenotype. Earliness, sometimes referred to as “early pistillate flowering,” is measured in days from time of seeding or transplanting to first appearance of pistillate flowers. This early flowering is a measure of the maturation of a plant, and results in a plant with fruit ready for market sooner than plant lines that do not exhibit the trait for earliness. Another aspect of the earliness in the inbred multiple disease resistant plant of the present invention is that it produces many early pistillate flowers, i.e., it is highly prolific. These traits have commercial value for the producer, and thus are desirable.

Incorporation of resistance to a single pathogen is accomplished via selection over one or more generations. In contrast, breeding for multiple pathogen resistance presents three other possible approaches to selection, including tandem selection, independent culling levels, and the selection index. Turner et al., Quantitative Genetics in Sheep Breeding, Cornell University Press, Ithaca, N.Y. (1967), which is hereby incorporated by reference in its entirety. These techniques were developed primarily for quantitative traits and are used regularly in most plant and animal breeding programs; they also provide the theoretical foundation for the selection of qualitatively inherited traits. These principles are well-known to those skilled in the art.

In breeding for multiple resistances, the biology of the different pathogens and their interactions with the host species affects the choice of a selection technique. Cross protection, synergism, variations in symptomatology, and escapes influence selection for multiple pathogen resistance. For example, cross protection resulting from simultaneous inoculations of closely related viruses can confound identification of resistant and susceptible phenotypes, and affect the accuracy of selection. In addition, the interaction of different viruses with the different genetic backgrounds of the host may cause symptomatology to vary, becoming an unreliable criteria for selection. Synergism among pathogens may affect the ability to detect pathogenicity. Lastly, escapes affect all selection techniques, but can be detected with repeated inoculations within a generation or over successive generations. Those skilled in the art will appreciate how these selection methods and others can be applied to practice the present invention.

In addition to traditional plant breeding techniques, cucumber breeding lines and cultivars may be developed using plant tissue culture techniques well known in the art. The term “tissue culture,” as used in the present application, is meant to be broad in scope and related to the culture of plant seeds, organs, explants, tissues, cells, or protoplasts in vitro or under sterile conditions. Tissue culture techniques include, but are not limited to, the following: micropropagation; meristem culture; somatic embryogenesis; somaclonal variation; in vitro selection; protoplast culture; somatic hybridization; and double-haploid breeding systems.

Various tissue culture techniques have been shown to produce plant germplasm with enhanced disease resistance (Daub, “Tissue Culture and the Selection of Resistance to Pathogens,” Ann. Rev. Phytopathology 24:159-186 (1986) and Hammerschlag, “In Vitro Approaches to Disease Resistance,” in Collins et al., eds., Applications of Genetic Engineering to Crop Improvement, Dordrecht: Matinus Nijhoff/Dr. W. Junk Publishers, pp. 453-490 (1984), which are hereby incorporated by reference in their entirety). Further, successful tissue cultures of cucurbitae using a variety of techniques have been reported in the art (Mackay et al., “Cucumis melo L. Callus Response to Toxins Produced by Myrothecium roridum Tode ex. Fries,” J. Amer. Soc. Hort. Sci. 119(2):356-360 (1994); Debeaujon et al., “Somatic Embryogenesis and Organogenesis from Protoplast-Derived Cultures of Muskmelon (Cucumis melo L.),” Acta Hort. (ISHS) 289:225-226 (1991); and Garcia-Sogo et al., “Enhancement of Morphogenetic Response in Cotyledon-Derived Explants of Cucumis melo Induced by Copper Ion,” Acta Hort. (ISHS) 289:229-230 (1991), which are hereby incorporated by reference in their entirety).

Several reference books are available that describe these and various other breeding methods suitable for carrying out the method of the present invention and for producing the resistant hybrid cucumber seeds and plants of the present invention, including, but not limited to, Allard, R. W., Principles of Plant Breeding, New York: Wiley (1960); Simmonds, N. W., ed., Evolution of Crop Plants, London: Longman (1979); Simmonds, N. W., ed., Principles of Crop Improvement, London, New York: Longman (1979); Sneep et al., eds., Plant Breeding Perspectives Wageningen, Neth.: Centre for Agricultural Publishing and Documentation (1979); and Fehr et al., eds., Principles of Cultivar Development, New York: Macmillan (1987), which are hereby incorporated by reference in their entirety. The information in the recommended texts, as well as those techniques known to those skilled in the art, can be used to carry out the present invention.

An example of a multiple disease resistant, early-flowering plant produced by the present invention is the cucumber cultivar designated ‘Marketmore 2000’(‘M2000’). ‘Marketmore 2000,’ the result of the cross between ‘Marketmore 97’and ‘Marketmore 421’ (see Example 1), yields an exemplary cucumber variety of the present invention exhibiting early flowering in addition to resistance to TLS, CMV, ZYMV, WMV, PRSV, cucumber scab, powdery mildew, downy mildew, Alternaria leaf blight, and Ulocladium leafspot disease. The cultivar ‘Marketmore 2000’ is generally characterized by phenotype as shown in Table 1, below.

TABLE 1

| | | Marketmore 2000 |
|---|---|---|
| Characteristic | | Phenotype |
| Predominate usage | | Slicing, Fresh Market |
| Predominate culture | | Outdoor |
| Days from seeding to market maturity | | 58 days |
| Habit | | Vine |
| Growth type | | Indeterminate |
| Sex | | Monoecious |
| Length of main stem (average of 25 plants) | | 115 cm |
| Internode Length, main stem (average of 25 | | 4.9 cm |
| plants) | | |
| Number of Nodes from Cotyledon leaves to Node | | 2 |
| bearing the first pistillate flower | | |
| Surface of plant mainstem | | Grooved, Ridged |
| Leaf length (measurements from mature blade of | | 181 mm |
| third leaf; average of 25 plants) | | |
| Leaf width (measurements from mature blade of | | 195 mm |
| third leaf average of 25 plants) | | |
| Petiole length (measurements from mature blade | | 12 cm |
| of third leaf; average of 25 plants) | | |
| Leaf color | | Dark green |
| Flower color | | Yellow |
| Fruit at Maturity: | Average Length | 20 cm |
| | Medial Diameter | 5 cm |
| | Average Weight | 248 gms |
| | Skin color | Predominately medium |
| | | green at blossom end; |
| | | dark green at stem |
| | | end |
| | Stripes | NA |
| | Skin texture | Thick, tough, not |
| | | ribbed, dull, coarse |
| | Spines | White, Few |
| | Fruit shape | Not necked |
| | End Tapered | Both Ends |
| | Stem end cross- | Circular |
| | section | |
| | Medial cross | Circular |
| | section | |
| | Blossom end | Circular |
| | cross-section | |
| | Tubercles (warts) | Few, obscure |
| Fruit at seed harvest maturity: | Length | 23 cm |
| | Medial diam. | 6 cm |
| | Color | Yellow, not striped |
| | Surface | Rough |
| | Netting | Slight or none |
| Fruit Setting | | Normally with Seeds |
| Seeds: | Average No. per | 260 |
| | fuit | |
| | Gram per 1,000 | 25 |
| | seeds | |

| | |
|---|---|
| Insect Resistance | Eastern Striped |
| | Cucumber Beetle |
| | (Acolymma vittata) |
| | Spotted Cucumber |
| | Beetle (Diabrotica |
| | undecipunctata |
| | howardi) |
| Disease resistance: | Target Leafspot |
| | (Corynespora |
| | cassiicola); |
| | Zucchini Yellow |
| | Mosaic Virus |
| | (ZYMV); |
| | Watermelon Mosaic |
| | Virus (WMV); |
| | Papaya Ringspot Virus |
| | (PRSV); |
| | Cucumber Mosaic |
| | Virus (CMV); |
| | Alternaria Leaf Blight |
| | (Alternaria |
| | cucumerina); |
| | Ulocladium Leafspot |
| | (Ulocladium |
| | cucurbitae) |
| | Cucumber Scab |
| | (Cladosporium |
| | cucerinum); |
| | Powdery Mildew |
| | (Erysiphe |
| | chicoracearum; |
| | Downy Mildew |
| | (Pseudoperonospora |
| | cubensis) |

The above description of the ‘Marketmore 2000’ plant in no way is meant to limit the phenotypic characteristics of the multiple disease resistant, early-flowering cucumber produced by the method of the present invention.

This invention also relates to the seeds of cucumber plant designated ‘Marketmore 2000,’ to the plants grown from the seeds of a cucumber designated ‘Marketmore 2000,’ and to methods for producing a cucumber plant produced by crossing the cucumber designated ‘Marketmore 2000’ with itself or another cucumber variety. All plants produced using the cucumber cultivar designated ‘Marketmore 2000’ as a parent are within the scope of this invention. Advantageously, the cucumber variety could be used in crosses with other, different, cucumber plants to produce first generation (F1) cucumber hybrid seeds and plants with superior characteristics. Thus, any such methods using the cucumber cultivar of the present invention are encompassed by this invention, including, but not limited to, pedigree breeding, selfing, intercrossing, backcrossing, crosses to populations, and the like. This includes tissue culture of regenerable cells of the inbred disease resistant, early-flowering plant designated herein as ‘Marketmore 2000,’ such that the tissue culture regenerates multiple progeny plants capable of expressing all the morphological and physiological characteristics of the hybrid plant. Plant tissue capable of such regeneration include leaves, pollen, embryos, roots, stems, root tips, anthers, flowers, seeds, and fruit. The breeding methods encompassing tissue culture regeneration are well known to those skilled in the art (Daub, “Tissue Culture and the Selection of Resistance to Pathogens,” Ann. Rev. Phytopathology 24:159-186 (1986) and Hammersehlag, “In Vitro Approaches to Disease Resistance,” in Collins et al., eds., Applications of Genetic Engineering to Crop Improvement, Dordrecht: Matinus Nijhoff/Dr. W. Junk Publishers, pp. 453-490 (1984), which are hereby incorporated by reference in their entirety).

### EXAMPLES

### Example 1

### Origin and Breeding of ‘Marketmore 2000’

The exemplary multiple disease resistant, early-flowering inbred cucumber seed and plant of the present invention is the cultivar known as ‘Marketmore 2000’ (‘M2000’), a sample of said seed having been deposited under ATCC accession number ______. ‘M2000’ was bred by crossing the inbred cucumber line ‘Marketmore 97’ once to the inbred cucumber line ‘Marketmore 421 ’ to produce a first generation hybrid. The ‘M97’ variety is characterized as monoecious, having uniform color and non-bitter fruit, and is resistant to the potyviruses (ZYMV, WMV, PRV), TLS, powdery and downy mildew, CMV, cucumber scab, and Alternaria leaf blight. In addition, cucumbers resistant to TLS are also expected to be resistant to Ulocladium leafspot disease (Zitter, et al., “A Leaf Spot of Cucumber Caused by Ulocladium cucurbitae in New York,” Plant Dis. 74:824-827 (1990), which is hereby incorporated by reference in its entirety). ‘M97’ was bred by crossing the inbred line ‘M87,’ which is resistant to TLS, CMV, and a number of other leafspot diseases, and the inbred line ‘M88,’ which is resistant to the potyviruses. Progeny of the ‘M87’×‘M88’ cross were selected for resistance to TLS and potyvirus. Selection was then done for yield and quality on lines showing combined resistance to TLS and the 3 potyviruses, resulting in ‘M97’.

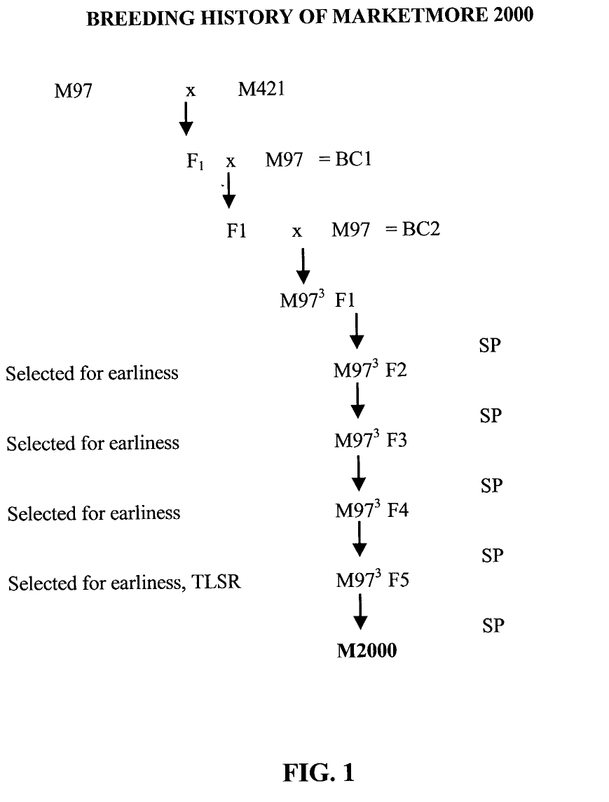

The ‘M421’ cucumber variety has all the resistances of ‘M97’ (i.e., resistance to the potyviruses and CMV, cucumber scab, and powdery and downy mildew), with the exception of Alternaria leaf blight and TLS resistance. In addition, ‘M421’ has the characteristic of early pistillate flowering (“early” or “earliness”). Two backcrosses of the F1 plant line from the ‘M97’×‘M421’ cross were made to ‘M97’ to ensure transfer of TLS resistance. Also, selections were made for earliness, which is a distinguishing characteristic of ‘M2000’ as compared to ‘M97’. FIG. 1 shows the origin and breeding history of ‘M2000’.

The result of combining the ‘M97’ trait of resistance to TLS, Ulocladium leafspot disease and Alternaria leaf blight, with the earliness trait of ‘M421,’ both parents having resistance to potyviruses, cucumber mosaic virus, scab, powdery mildew, and downy mildew, is a multiple disease resistant, early flowering cucumber line of high commercial value.

### Example 2

### Distinctiveness of ‘Marketmore 2000’

‘Marketmore 2000’is similar to parental ‘M97’ in plant vigor and habit; leaf shape and size; and fruit shape, size and skin color.

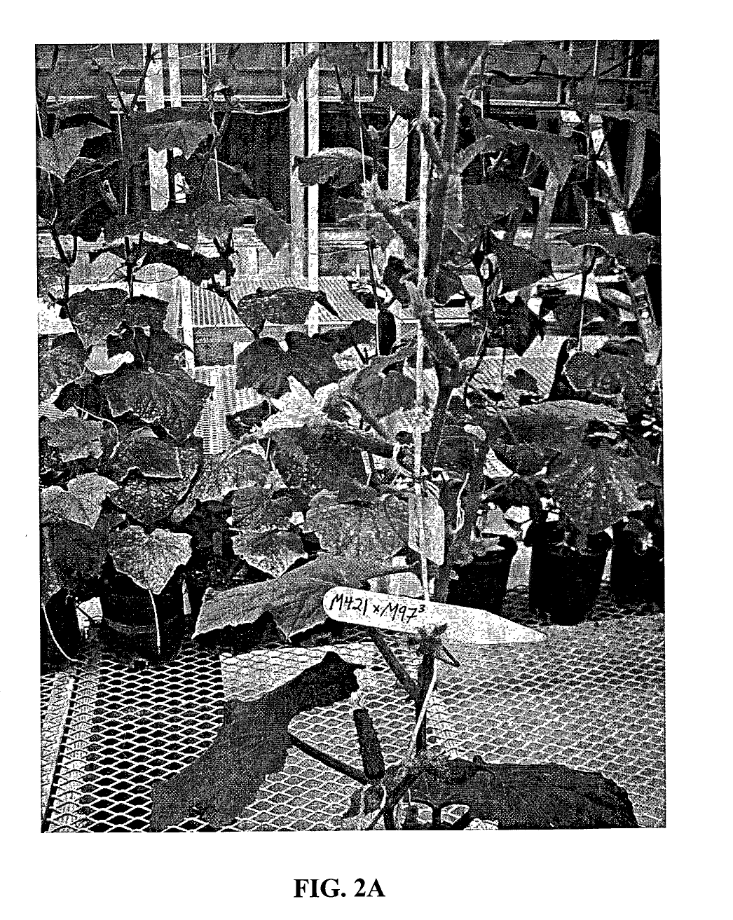

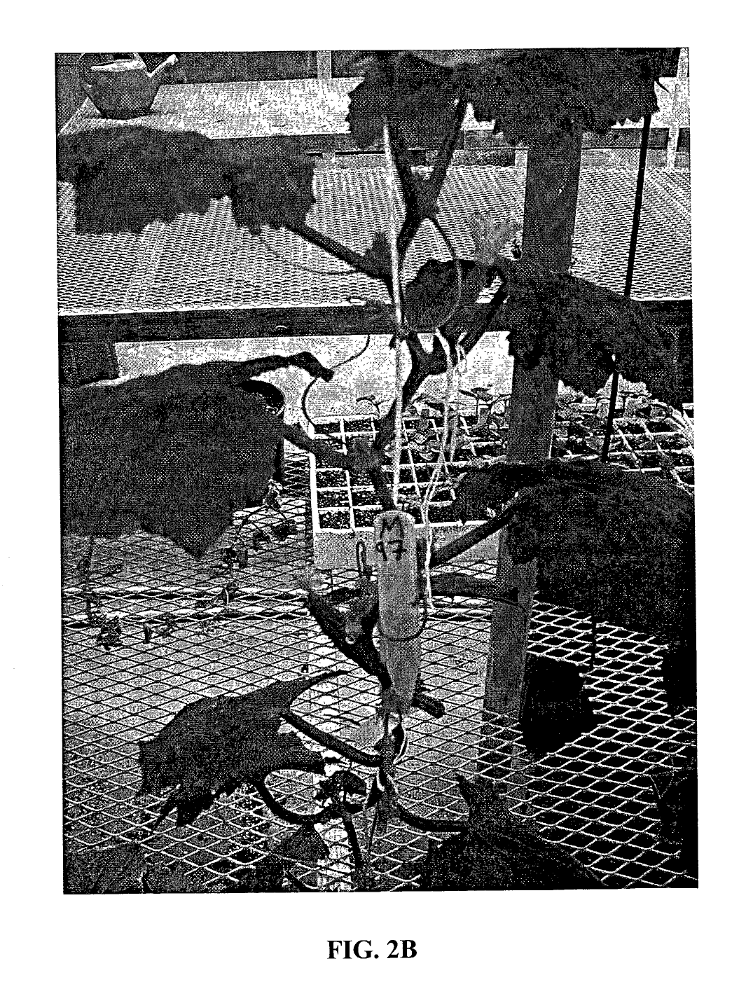

‘Marketmore 2000’ retains the resistances to TLS, PVR, CMV, cucumber scab, Ulocladium leafspot disease, powdery mildew, and downy mildew of ‘M97’ and has, additionally, the earliness trait of ‘M421’. Compared to ‘M97,’ ‘M2000’ produces pistillate flowers earlier, though the pistillate flowers may be on the same nodes. ‘M2000’ also produces more pistillate flowers than ‘M97’. The prolific flowering of the ‘M 2000’ line is shown in FIG. 2A in comparison to the flowering of its parental line ‘Marketmore 97,’ shown in FIG. 2B.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. An early flowering, multiple disease resistant inbred cucumber seed having resistance to target leafspot disease, zucchini yellow mosaic virus, watermelon mosaic virus, and papaya ringspot virus, a sample of said seed having been deposited under ATCC accession number ______.

2. An early flowering, multiple disease resistant cucumber plant, or parts thereof, produced by growing the seed of claim 1.

3. Pollen of the plant according to claim 2.

4. An ovule of the plant according to claim 2.

5. A tissue culture of regenerable cells of an inbred cucumber plant according to claim 2, wherein the tissue regenerates plants capable of expressing all the morphological and physiological characteristics of an inbred cucumber plant.

6. A tissue culture according to claim 5, wherein the tissue culture is produced from a plant part selected from the group consisting of leaves, pollen, embryos, roots, tip, anthers, and flowers.

7. A cucumber plant regenerated from the tissue culture according to claim 5.

8. A method for producing an early flowering, multiple disease resistant inbred cucumber seed comprising:
providing a first inbred C. sativus L. cucumber plant line which is resistant to target leafspot disease, zucchini yellow mosaic virus, watermelon mosaic virus, and papaya ringspot virus;
providing a second inbred C. sativus L. cucumber plant line which is early flowering and resistant to zucchini yellow mosaic virus, watermelon mosaic virus, and papaya ringspot virus;
crossing the first and second inbred C. sativus L. cucumber plant lines to yield an F1 hybrid C. sativus L. cucumber line which is early flowering and resistant to target leafspot disease;
backcrossing the F1 hybrid C. sativus L. cucumber line to yield an early flowering inbred cucumber plant line which is resistant to target leafspot disease, zucchini yellow mosaic virus, watermelon mosaic virus, and papaya ringspot virus resistant; and
harvesting the seeds therefrom.

9. The method according to claim 8, wherein the first inbred cucumber is additionally resistant to cucumber scab, powdery mildew, downy mildew, Alternaria leaf blight, Ulocladium leafspot disease, and cucumber mosaic virus.

10. The method according to claim 8, wherein the second inbred cucumber is additionally resistant to cucumber scab, powdery mildew, downy mildew, and cucumber mosaic virus.

11. The method according to claim 8, wherein the first inbred C. sativus L. cucumber plant is selected from the ‘M97’ line.

12. The method according to claim 8, wherein the second inbred cucumber plant is selected from the ‘M421’ line.

13. A method according to claim 8, further comprising:
growing the inbred seed to yield an early flowering inbred cucumber plant which is resistant to target leafspot disease, zucchini yellow mosaic virus, watermelon mosaic virus, and papaya ringspot virus.

14. The method according to claim 13, further comprising:
using germplasm derived from the early flowering inbred cucumber plant which is resistant to target leafspot disease, zucchini yellow mosaic virus, watermelon mosaic virus, and papaya ringspot virus to a plant breeding program to yield multiple disease resistant, early flowering cucumber inbred seeds, wherein said breeding program comprises traditional plant breeding techniques selected from the group consisting of pedigree breeding, selfing, intercrossing, and backcrossing.

15. The method according to claim 14, wherein the early flowering inbred cucumber plant is resistant to target leafspot disease, zucchini yellow mosaic virus, watermelon mosaic virus, papaya ringspot virus, Alternaria leaf blight, Ulocladium leafspot disease, cucumber scab, powdery mildew, downy mildew, and cucumber mosaic virus,

16. An early flowering, multiple disease resistant inbred cucumber seed produced by the method according to claim 8.

17. An early flowering, multiple disease resistant inbred cucumber plant, or parts thereof, produced by the method according to claim 8.

18. Pollen of the plant according to claim 17.

19. An ovule of the plant according to claim 17.

20. A method for developing a cucumber plant in a cucumber plant breeding program comprising:
subjecting the cucumber plant or parts thereof according to claim 2 to a plant breeding program.

21. A method according to claim 20, wherein the plant breeding program comprises techniques selected from the group consisting of pedigree breeding, selfing, intercrossing, and backcrossing.

22. An early flowering, multiple disease resistant cucumber plant, or parts thereof, produced by the method of claim 21.

23. A method of producing a hybrid cucumber seed comprising:
crossing the early flowering, multiple disease resistant inbred cucumber plant according to claim 2 with another, different cucumber plant.

24. A hybrid cucumber seed produced by the method of claim 23.

25. A hybrid cucumber plant, or parts thereof, produced by growing the hybrid cucumber seed according to claim 24.

26. Cucumber seed produced by growing said hybrid plant according to claim 24, and harvested therefrom.
